(19) United States
(12) Patent Application Publication
(10) Pub. No.: US 2019/0192111 A1
(43) Pub. Date: Jun. 27, 2019

(54) Ultrasonic Imaging Device and Operation Method Thereof

(71) Applicant: Hitachi, Ltd., Chiyoda-ku, Tokyo (JP)

(72) Inventors: Motochika SHIMIZU, Tokyo (JP); Tomohiko TANAKA, Tokyo (JP); Takashi OKADA, Tokyo (JP); Hajime SAKASHITA, Tokyo (JP); Yoshinori SEKI, Tokyo (JP)

(21) Appl. No.: 16/308,951

(22) PCT Filed: Jun. 13, 2017
(86) PCT No.: PCT/JP2017/021781

§ 371 Date: Dec. 11, 2018

(30) Foreign Application Priority Data
Jul. 19, 2016 (JP) 2016-141217

(51) Int. Cl. A61B 8/06; A61B 8/08; A61B 8/00; A61B 8/14

(52) U.S. Cl. CPC A61B 8/06; A61B 8/485; A61B 8/463; A61B 8/5292; A61B 8/488; A61B 8/5223; A61B 8/14

(57) ABSTRACT

The present invention provides a technique for estimating a blood flow velocity gradient in the vicinity of a blood vessel wall surface with high accuracy under various blood flow conditions. In an ultrasonic imaging device and an operation method thereof for calculating an estimated value of the blood flow velocity gradient on a wall surface in a blood vessel of an examination target from an echo signal reflected by the examination target, a blood flow velocity gradient distribution is calculated from a blood flow velocity in a direction parallel to a blood vessel wall surface at a plurality of measurement points calculated from the echo signal in a radial direction from the blood vessel wall surface toward the center of the blood vessel, a predetermined range is calculated in the calculated blood flow velocity gradient distribution, and the estimated value of the blood flow velocity gradient on the blood vessel wall surface is calculated from values of the blood flow velocity and the blood flow velocity gradient at the measurement points within the calculated predetermined range.

### TECHNICAL FIELD

The present invention relates to a medical ultrasonic imaging device, and relates to a technique for obtaining a blood flow velocity gradient in the vicinity of a blood vessel wall surface.

### BACKGROUND ART

One of the major causes of death in developed countries is cardiovascular diseases, such as heart failure, and many of which are related to arteriosclerosis. It is pointed out that the rate of progression of arteriosclerosis varies depending on the magnitude of the stimulation from a blood flow with respect to the blood vessel wall surface. Therefore, a wall shear stress that acts on the blood vessel wall surface is attracting attention as a diagnosis index of early arteriosclerosis.

One method of calculating the wall shear stress is a calculation method based on a blood flow velocity distribution measured by an ultrasonic Doppler method using an ultrasonic imaging device. In the method, it is required to obtain a blood flow velocity gradient which is a spatial differential of the blood flow velocity distribution with high precision in the vicinity of the blood vessel wall surface.

In the ultrasonic Doppler method, movement velocity information of a reflector can be obtained from an echo signal using the Doppler effect, but the reflector includes not only the red blood cells in the blood flow but also surrounding tissues, such as a blood vessel membrane or muscle. Since the blood flow in the vicinity of the blood vessel wall surface is as low as the movement of the surrounding tissue due to friction with the blood vessel wall surface, the echo signal of the blood flow is buried in a signal, from the surrounding tissues or the like, other than that of the detection target, and the measurement accuracy of the blood flow velocity deteriorates in the vicinity of the blood vessel wall surface. Therefore, by simply differentiating the blood flow velocity distribution measured by the ultrasonic imaging device, the blood flow velocity gradient in the vicinity of the blood vessel wall surface cannot be obtained with high accuracy, and the accuracy with which early clinical diagnoses of arteriosclerosis is possible is not realized.

The ultrasonic imaging method aimed at improving measurement accuracy of the blood flow velocity gradient includes the technique described in NPL 1. In NPL 1, in “B. Velocity reconstruction and WSR measurement” of “II. MATERIALS AND METHOD”, it is described that “In the first step the two regions that extend for about 5-10% of the diameter from the wall positions towards the vessel lumen are located. The profile measured in these regions is substituted by a line that starts at the wall with velocity 0 and ramps up to join the remaining—measured—profile”. In this method, the blood flow velocity distribution to the region that extends 5-10% of the blood vessel diameter from the blood vessel wall surface is rejected, interpolation is performed with respect to the rejected region, and then the blood flow velocity gradient in the vicinity of the blood vessel wall surface is estimated.

### CITATION LIST

### Non-Patent Literature

NPL 1: Improved Wall Shear Rate Method for Robust Measurements (Stefano Ricci et al., 2014 IEEE International Ultrasonics Symposium Proceedings, 432-435, 2014.)

### SUMMARY OF INVENTION

### Technical Problem

In NPL 1, a method of determining a region for rejecting measured values of the blood flow with reference to a blood vessel diameter is applied, and the method is verified by a simulation using a carotid artery as a model. However, since the actually measured blood flow velocity distribution varies depending on the shape or flexibility of the blood vessel that is an examination target, the properties of the vascular endothelium, the beat and the like, in the method of determining the rejected region with reference only to the blood vessel diameter, depending on the blood flow condition, a case where the measured value of the blood flow velocity which is buried in the signal other than that of the detection target is not sufficiently rejected is remained. As a result, there is a possibility of using measured values of the blood flow velocity with low reliability for estimating the blood flow velocity gradient in the vicinity of the blood vessel wall surface.

An object of the present invention is to provide an ultrasonic imaging device and an operation method thereof for estimating the blood flow velocity gradient in the vicinity of the blood vessel wall surface with high accuracy even under various blood flow conditions having different shapes of the blood vessel, flexibility, the properties of the vascular endothelium, the beat and the like.

### Solution to Problem

For solving the above-described problem, in the present invention, there is provided an ultrasonic imaging device including: a receiving unit that receives an echo signal reflected by an examination target; and a signal processing unit that processes the echo signal received by the receiving unit, in which the signal processing unit includes a velocity gradient distribution operation unit that calculates a blood flow velocity gradient distribution from a value of a blood flow velocity, in a direction parallel to a blood vessel wall surface of the examination target, calculated from the echo signal at a plurality of measurement points arranged in a radial direction from the blood vessel wall surface toward the center of a blood vessel, a range specifying operation unit that calculates a predetermined range in the blood flow velocity gradient distribution, and a wall surface velocity gradient operation unit that calculates an estimated value of a blood flow velocity gradient on the blood vessel wall surface from values of a blood flow velocity and the blood flow velocity gradient at the measurement points within the predetermined range.

In addition, for solving the above-described problem, in the present invention, there is provided an operation method in an ultrasonic imaging device, the method including: a step of extracting a blood flow velocity component in an ultrasonic irradiation direction in a blood vessel of an examination target from an echo signal reflected by the examination target; a step of calculating a blood flow velocity distribution which is a distribution of blood flow velocities in a direction parallel to a blood vessel wall surface at a plurality of measurement points arranged in a radial direction from the blood vessel wall surface toward the center of a blood vessel, based on the blood flow velocity component; a step of calculating a blood flow velocity gradient distribution from the blood flow velocity distribution by a differential operation; a step of calculating a predetermined range in the blood flow velocity gradient distribution; a step of selecting one or more measurement points among the measurement points within the predetermined range; and a step of calculating an estimated value of the blood flow velocity gradient on the blood vessel wall surface from values of the blood flow velocity and the blood flow velocity gradient at the selected measurement points.

### Advantageous Effects of Invention

According to the present invention, it is possible to estimate a blood flow velocity gradient in the vicinity of a blood vessel wall surface with high accuracy under various blood flow conditions.

### BRIEF DESCRIPTION OF DRAWINGS

FIG. 1 is a block diagram illustrating a configuration example of an ultrasonic imaging device according to Example 1.

FIG. 2 is a view illustrating a computation processing flow illustrating an embodiment of an operation of a signal processing unit in Example 1.

FIG. 3 is a view illustrating an example of a blood flow velocity distribution and an example of a blood flow velocity gradient distribution.

FIG. 4 is a view illustrating an approximate model of the blood flow velocity gradient distribution used for a wall surface velocity gradient operation and a position of a blood vessel wall surface.

FIG. 5 is a view illustrating an example of a display image in a predetermined range formed in a display image forming unit.

FIG. 6 is a view illustrating an example of a display image of a spatial distribution of a wall shear stress formed in the display image forming unit.

FIG. 7 is a diagram illustrating an example of a display image of a time-series change in the wall shear stress formed in the display image forming unit.

FIG. 8 is a view illustrating a computation processing flow illustrating an embodiment of an operation of a signal processing unit in Example 2.

FIG. 9 is a view illustrating a computation processing flow illustrating an embodiment of an operation of a signal processing unit in Example 3.

FIG. 10 is a view illustrating an integration range of a blood flow rate operation in Example 3.

### DESCRIPTION OF EMBODIMENTS

Hereinafter, examples of the present invention will be described below with reference to the drawings.

### Example 1

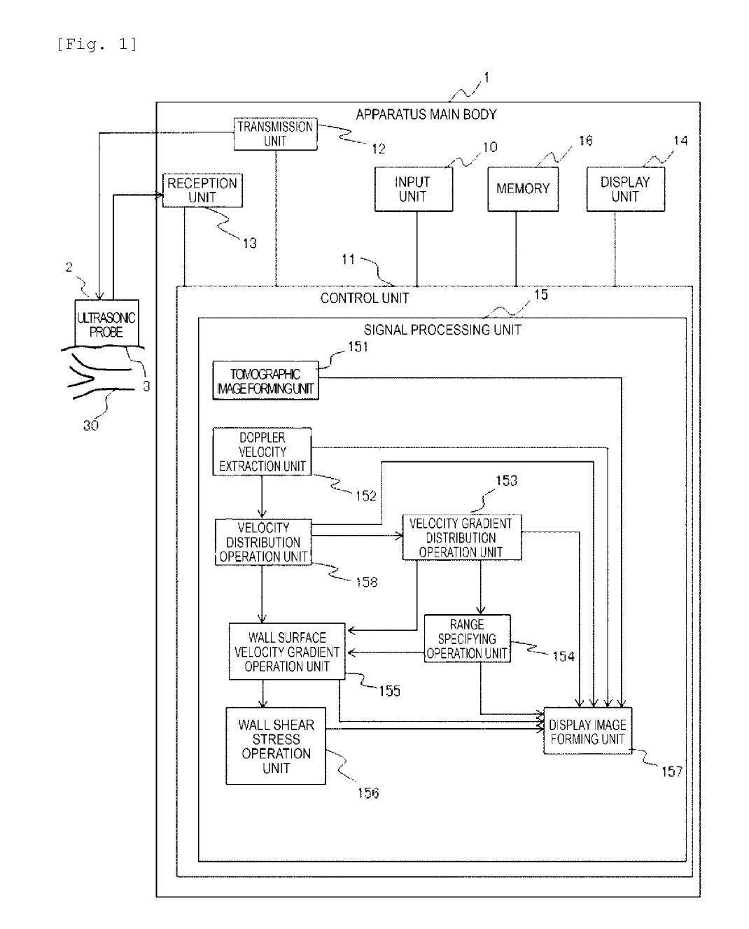

FIG. 1 is a block diagram illustrating a configuration example of an ultrasonic imaging device having a wall shear stress measuring function according to Example 1. The wall shear stress is a blood flow stimulus that causes changes in vascular endothelial cells and is a parameter that attracts attention in clinical research as a diagnosis index in early diagnosis of arteriosclerosis. In addition, the magnitude of the wall shear stress affects the breakdown of the plaque generated by advanced arteriosclerosis, and is also deeply related to risk prediction of plaque breakdown.

An apparatus main body 1 is for generating an ultrasonic image while controlling an ultrasonic probe 2, and includes an input unit 10, a control unit 11, a transmission unit 12 that transmits an ultrasonic signal, a reception unit 13 for receiving an echo signal, a display unit 14, a signal processing unit 15, and a memory 16.

The ultrasonic probe 2 is in contact with a living body 3 of an examinee and irradiates a blood vessel 30 in the living body 3 with ultrasonic waves in accordance with the signal generated by the transmission unit 12 and the reception unit 13 receives the echo signal of the blood vessel 30. The ultrasonic probe 2 generates a continuous wave or a pulse wave in accordance with a scanning method. Further, according to the scanning method of the ultrasonic probe 2, a planar imaging method for imaging a two-dimensional section or a stereoscopic imaging method for imaging a three-dimensional region may be appropriately selected.

The function of each configuration element of the apparatus main body 1 will be described. The input unit 10 includes a keyboard and a pointing device for setting the operating conditions of the ultrasonic imaging device with respect to the control unit by physicians or technicians (hereinafter, collectively referred to as examiners) who operate the ultrasonic imaging device. In addition, in a case where information from external equipment, such as electrocardiogram, is used for examination, a function of capturing information from the external equipment is also provided.

The control unit 11 controls the transmission unit 12, the reception unit 13, the display unit 14, and the signal processing unit 15 based on the operation conditions of the ultrasonic imaging device set by the input unit 10, and can be built in, for example, a central processing unit (CPU) of a computer system.

The transmission unit 12 includes an oscillator that generates a signal of a predetermined frequency and sends a driving signal to the ultrasonic probe 2. Although not illustrated, the reception unit 13 includes a reception circuit and an analog-to-digital (A/D) converter having a sampling frequency of usually 10 MHz to 50 MHz, and in addition to this, also performs signal processing, such as phase addition, detection, or amplification, with respect to the echo signal received by the ultrasonic probe 2. The processing includes a filter (hereinafter, referred to as a wall filter) that eliminates a low velocity components included in a typical ultrasonic imaging device. Here, instead of the reception unit 13, the A/D converter may be provided in front of the signal processing unit 15, and in this case, the signal processing unit 15 performs signal processing, such as phase addition, detection, amplification, or wall filter. In addition, although not illustrated, the reception unit 13 may have a received data memory for temporarily storing the echo signals for each receiving element of the ultrasonic probe 2 or for each opening portion bundling the elements.

Next, the detailed configuration elements of the signal processing unit 15 will be described. Here, a case where the signal processing unit 15 is realized by software executed by the CPU will be described as an example. The signal processing unit 15 includes a tomographic image forming unit 151, a Doppler velocity extraction unit 152, a velocity distribution operation unit 158, a velocity gradient distribution operation unit 153, a range specifying operation unit 154, a wall surface velocity gradient operation unit 155, a wall shear stress operation unit 156, and a display image forming unit 157 as main elements, the CPU reads and executes the program, and accordingly, functions which will be described later are realized.

The tomographic image forming unit 151 forms a two-dimensional tomographic image of tissues in an irradiation region of the examination target or a three-dimensional tomographic image of tissues by using a two-dimensional array probe or a mechanical probe, from the echo signal output from the reception unit 13. The Doppler velocity extraction unit 152 extracts a blood flow velocity component (hereinafter, referred to as “Doppler velocity”) in an ultrasonic irradiation direction from the echo signal output from the reception unit 13. At this time, the Doppler velocity is obtained as a two-dimensional spatial distribution by using a planar imaging method, or as a three-dimensional spatial distribution by using a stereoscopic imaging method.

The velocity distribution operation unit 158 calculates the blood flow velocity (hereafter, in a case where the direction is not particularly illustrated, the blood flow velocity indicates the blood flow velocity in the direction along the wall surface of the blood vessel 30) in the direction along the wall surface of the blood vessel 30 with respect to the Doppler velocity output by the Doppler velocity extraction unit 152, and calculates the blood flow velocities at a plurality of measurement points arranged in the radial direction from the wall surface of the blood vessel 30 toward the center of the blood vessel.

The velocity gradient distribution operation unit 153 calculates the blood flow velocity gradient distribution by a differential operation from the blood flow velocity distribution calculated by the velocity distribution operation unit 158. The range specifying operation unit 154 calculates a predetermined range for the blood flow velocity gradient distribution output by the velocity gradient distribution operation unit 153.

The wall surface velocity gradient operation unit 155 calculates the blood flow velocity gradient (hereinafter, referred to as wall surface velocity gradient) on the blood vessel wall surface by using the values of the blood flow velocity and the blood flow velocity gradient at the measurement point within the predetermined range calculated by the range specifying operation unit 154.

The wall shear stress operation unit 156 configures a diagnosis index operation unit that calculates information which is a diagnosis index of the examination target. The wall shear stress operation unit 156 calculates the wall shear stress that acts on the blood vessel wall surface by multiplying a viscosity coefficient of blood given as a predetermined value, a value input from the input unit 10, or a value calculated from the echo signal with respect to the value of the wall surface velocity gradient calculated by the wall surface velocity gradient operation unit 155. The display image forming unit 157 includes a scan converter and forms a display image to be displayed on the display unit 14. The display image to be formed includes information, such as the tomographic image formed by the tomographic image forming unit 151, the Doppler velocity extracted by the Doppler velocity extraction unit 152, the blood flow velocity distribution calculated by the velocity distribution operation unit 158, the blood flow velocity gradient distribution calculated by the velocity gradient distribution operation unit 153, the predetermined range calculated by the range specifying operation unit 154, the wall surface velocity gradient calculated by the wall surface velocity gradient operation unit 155, the wall shear stress calculated by the wall shear stress operation unit 156, and the like.

In addition, the functions of a part or the entirety of the configuration elements of the signal processing unit 15 may be realized as well as by the same CPU as that which configures the control unit 11 or software executed by a different CPU, by hardware, such as an application specific integrated circuit (ASIC), a field-programmable gate array (FPGA), or graphics processing unit (GPU).

The memory 16 stores echo signals, information (information and the like instructed by the examiner by the input unit 10) necessary for the operation in the signal processing unit 15 or processing results (tomographic image, Doppler velocity, blood flow velocity distribution, blood flow velocity gradient distribution, predetermined range in blood flow velocity gradient distribution, wall surface velocity gradient, wall shear stress, display image, and the like) of the signal processing unit 15.

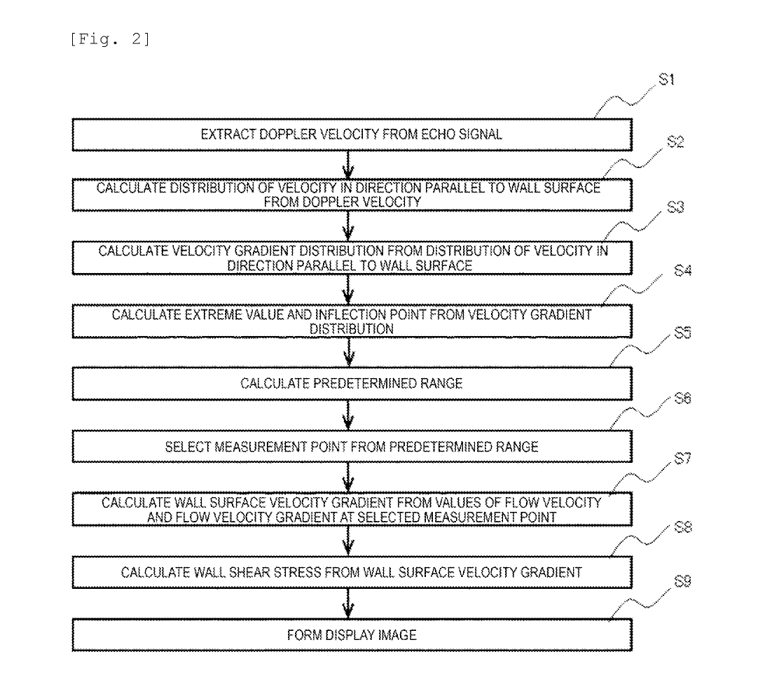

Based on the configuration of the above-described apparatus, an example of the embodiment of the operation of the signal processing unit 15 will be described with reference to a computation processing flow illustrated in FIG. 2.

<Step S1>

After receiving the echo signal output by the reception unit 13, the Doppler velocity extraction unit 152 applies the wall filter to extract blood flow information and extracts the Doppler velocity from the echo signal using the color Doppler method. At this time, a blood flow velocity measurement method, such as a pulse wave Doppler method, may be used. Further, various filters, such as a low-pass filter, a band-pass filter, and a smoothing filter, may be applied to the spatial distribution or the time change of the extracted Doppler velocity to obtain the desired distribution or the time change.

<Step S2>

After receiving the Doppler velocity extracted by the Doppler velocity component extraction unit, the velocity distribution operation unit 158 calculates the blood flow velocity in the direction along the wall surface of the blood vessel 30. At this time, first, a velocity vector of the blood flow is calculated by using a vector Doppler method which calculates the velocity vector from the Doppler velocity obtained at angles in two or more directions, and then, the velocity component in the direction along the wall surface of the blood vessel 30 of the velocity vector is obtained. In addition, the method of obtaining the blood flow velocity in the direction along the blood vessel wall surface is not limited to the vector Doppler method, and for example, the extracted Doppler velocity may be estimated by considering the ultrasound irradiation angle or the flow direction of the blood flow. After this, from the calculated blood flow velocity, a blood flow velocity distribution which is a distribution of the blood flow velocities at the plurality of measurement points arranged in the radial direction from the wall surface of the blood vessel 30 toward the center of the blood vessel is calculated. At this time, the radial direction of the blood vessel 30 may be automatically computed from the tomographic image, such as a B mode image formed by the tomographic image forming unit 151 and stored in the memory 16, or may be instructed by the examiner through the input unit 10.

<Step S3>

The velocity gradient distribution operation unit 153 calculates the blood flow velocity gradient distribution by a differential operation after receiving the blood flow velocity distribution calculated by the velocity distribution operation unit 158. At this time, various filters, such as a low-pass filter, a band-pass filter, and a smoothing filter, may be applied to the calculated blood flow velocity gradient distribution to obtain a desired distribution.

<Step S4>

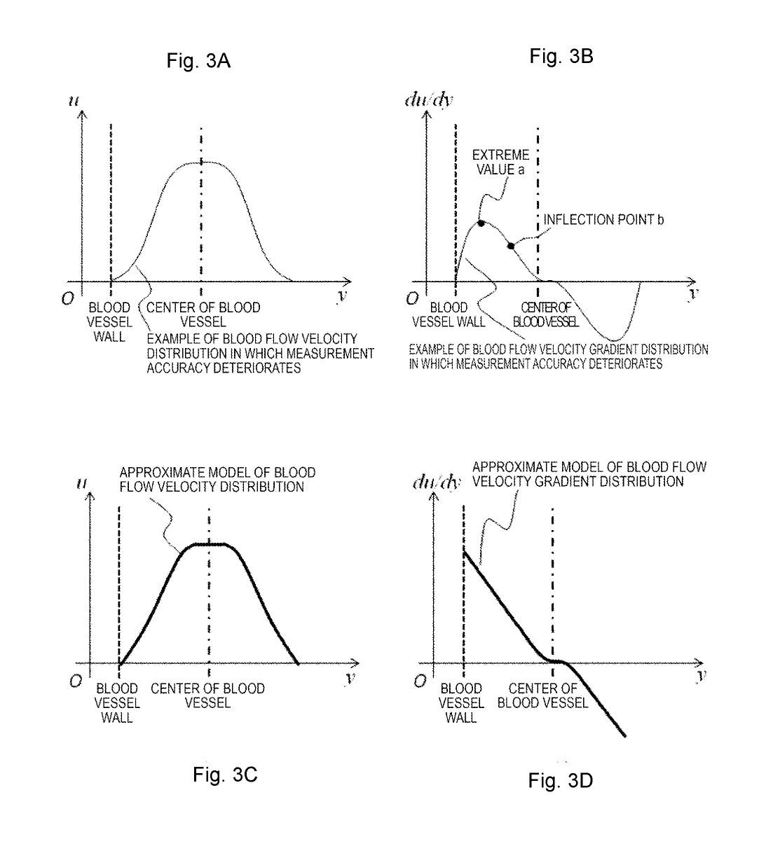

After receiving the blood flow velocity gradient distribution calculated by the velocity gradient distribution operation unit 153, the range specifying operation unit 154 first calculates at least one of an extreme value and an inflection point of the blood flow velocity gradient distribution. With reference to FIG. 3, the grounds which determine the region to be rejected in the blood flow velocity gradient distribution by using coordinates of at least one of the extreme value and the inflection point of the blood flow velocity gradient distribution will be described.

FIG. 3 (a) illustrates the measured values of the blood flow velocity at the plurality of measurement points arranged in the radial direction (y direction) from the blood vessel wall surface toward the center of the blood vessel as a distribution diagram, and FIG. 3(b) illustrates the blood flow velocity gradient distribution obtained by calculating the blood flow velocity distribution of FIG. 3 (a) by a differential operation. Here, the dotted line in the drawing indicates the position of the blood vessel wall surface, and the one dot chain line indicates the center of the blood vessel, respectively. Here, the distribution diagrams of FIGS. 3(a) and 3(b) are influenced by the wall filter.

Meanwhile, FIG. 3(c) is an example of an approximate model of the blood flow velocity distribution, and FIG. 3(d) is an example of an approximate model of the blood flow velocity gradient distribution. First, the approximate models will be described. The three-dimensional shape that most easily represents the blood vessel is a straight tube (hereinafter, simply referred to as a circular tube) of a circular section. Supposing that the blood flow is a normal flow having no time dependence, the blood flow velocity distribution is represented by a quadratic curve from the Hagen-Poiseuille flow equation of the equation (1).

$$u = - \frac{{R}^{2}}{4 μ} \frac{dp}{dx} ( 1 - \frac{{r}^{2}}{{R}^{2}} ) \qquad ( 1 )$$

Here, u represents the blood flow velocity, R represents the blood vessel radius, x represents the distance in the flow direction, and r represents the distance in the radial direction from the center of the blood vessel. Further, dp represents a change amount of a pressure p in a minute section dx at two locations in the flow direction, and μ represents a viscosity coefficient of the blood.

Since the blood flow velocity gradient distribution can be calculated as a spatial differential of the blood flow velocity distribution, the blood flow velocity gradient distribution of the circular tube is represented by a primary linear straight line from the equation of the equation (2).

$$\frac{du}{dr} = \frac{1}{2 μ} \frac{dp}{dx} r \qquad ( 2 )$$

In the actual blood flow, the blood flow velocity distribution does not become the same as the Hagen-Poiseuille flow since the shape of the complicated blood vessel, such as bending, and a time fluctuation of the blood flow, such as the beat, exist, but at a location in the vicinity of the blood vessel wall surface, the influence of the slipless condition of the wall surface is strong and is common to the Hagen-Poiseuille flow. Therefore, in FIGS. 3 (c) and 3 (d), the blood flow velocity distribution in the vicinity of the blood vessel wall surface is approximated by the quadratic curve, the blood flow velocity gradient distribution in the vicinity of the blood vessel wall surface is approximated by a primary linear line, and the blood flow velocity gradient distribution in the vicinity of the center of the blood vessel is convex downward and modeled as smoothly converging to 0. Here, the approximate model of the blood flow velocity distribution is not limited to the quadratic curve, but approximation by other functions, such as polynomials and exponential functions, is also possible.

Next, the blood flow velocity distribution and the blood flow velocity gradient distribution influenced by the wall filter illustrated in FIGS. 3(a) and 3(b) will be described. The wall filter is a filter for excluding the movement of surrounding tissues, such as the vascular membrane or muscle, which is slow compared to the blood flow. Therefore, due to the characteristics, the signal of the blood flow velocity in the vicinity of the blood vessel wall surface, which became slow due to the frictional force received from the blood vessel wall surface, is weakened, and as a result, the blood flow velocity is measured underestimately. Therefore, in the blood flow velocity gradient distribution obtained by performing the differential operation with respect to the blood flow velocity distribution after applying the wall filter, as illustrated in FIG. 3(b), a tendency in which a monotonous increase turns into a monotonous decrease as approaching the blood vessel wall surface from the center of the blood vessel, and the blood flow velocity gradient is converged to 0 on the blood vessel wall surface, is illustrated.

For the reasons described above, in the blood flow velocity gradient distribution illustrated in FIG. 3 (b), at least in a range to the blood vessel wall surface from the point (hereinafter, referred to as extreme value a) at which the monotonous increase turns into the monotonous decrease from the blood vessel wall surface toward the center of the blood vessel, the blood flow velocity and the blood flow velocity gradient are underestimated. In addition, among the inflection points of the blood flow velocity gradient distribution, the point (hereinafter, referred to as inflection point b) at which the upward convex turns into the downward convex is an index indicating that the blood flow velocity gradient distribution has a shape that conforms to the approximate model of FIG. 3 (d). Therefore, the extreme value a or the inflection point b of the blood flow velocity gradient distribution is an index of a region in which the blood flow velocity is underestimated due to the influence of the wall filter, that is, a region in which the measurement value of the blood flow velocity is buried in the signal other than that of the detection target, such as the surrounding tissue, under various blood flow conditions.

<Step S5>

The range specifying operation unit 154 subsequently determines a region to be rejected in the blood flow velocity gradient distribution with reference to at least one of the extreme value a and the inflection point b and calculates a region excluding the rejected region from the blood flow velocity gradient distribution, that is, a predetermined range in which the value on the distribution is used for the estimation of the blood flow velocity gradient on the wall surface of the blood vessel 30. In a case where the extreme value a is set as the boundary of the predetermined range, the value of the measurement point near the wall surface on the distribution can be used for the estimation of the wall surface velocity gradient. In addition, it is also possible to regard the part from the infection point b to the blood vessel wall surface as underestimated region due to the influence of the wall filter, and to set the inflection point b as the boundary of the predetermined range. In addition, a point which is set by the examiner with reference to the coordinates and has coordinates different from those may be the boundary of the predetermined range. The range to be calculated may be defined from the focused blood vessel wall surface to the center of the blood vessel, or may be defined from the blood vessel wall surface to the opposing blood vessel wall surface through the center of the blood vessel.

<Step S6>

After receiving the blood flow velocity distribution calculated from the velocity distribution operation unit 158, the blood flow velocity gradient distribution calculated from the velocity gradient distribution operation unit 153, and the predetermined range calculated from the range specifying operation unit 154, the wall surface velocity gradient operation unit 155 first selects the measurement point to be used for the operation of the wall surface velocity gradient from the predetermined range.

At this time, one or more measurement points to be selected may be selected according to a method of the wall surface velocity gradient operation which will be described later. Preferably, for the reasons below, two measurement points, that is, the extreme value a and the inflection point b, may be selected. By selecting the extreme value a, it is possible to use the values of the blood flow velocity and the blood flow velocity gradient at the measurement point which is the nearest to the wall surface within the predetermined range, and further, by selecting the inflection point b, it is possible to use the values of the blood flow velocity and the blood flow velocity gradient at the point at which the blood flow velocity gradient distribution becomes to have a shape that conforms to the approximate model in FIG. 3(d).

<Step S7>

The wall surface velocity gradient operation unit 155 subsequently calculates the estimated value of the wall surface velocity gradient by using the values of the blood flow velocity and the blood flow velocity gradient at the selected measurement point.

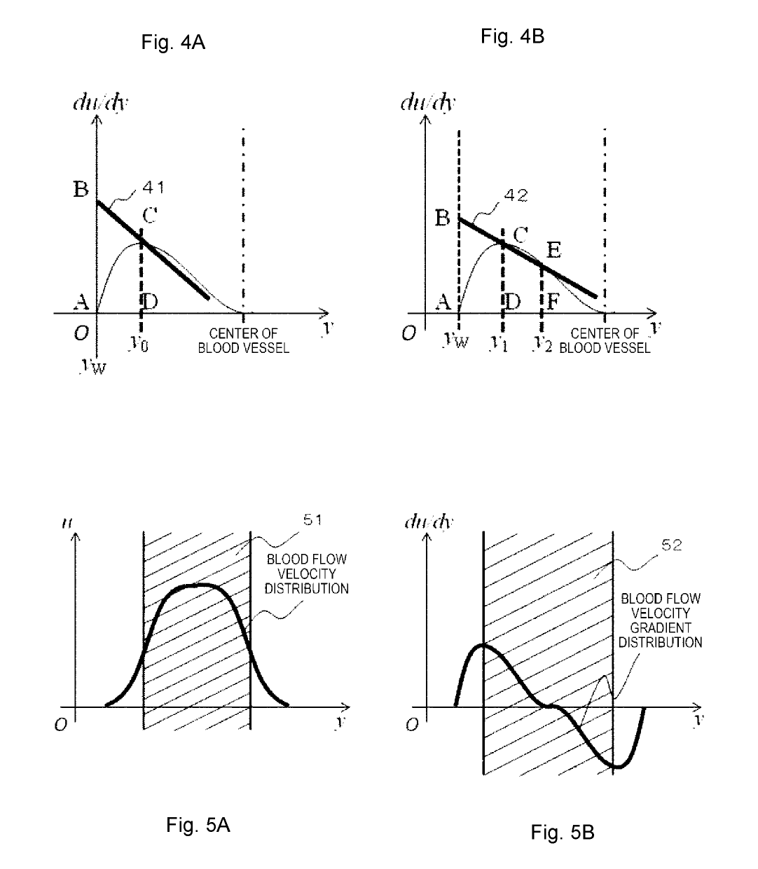

Here, as a representative example, description will be made with reference to FIG. 4 with respect to a case (a) where the selected measurement point is one point and a case (b) where the selected measurement points are two points. In FIG. 4, the range from the extreme value a to the center of the blood vessel is set as a predetermined range with the extreme value a as one of the boundaries and the other boundary as the center of the blood vessel. Here, the inner side of the predetermined range includes the boundary. Here, similar to in FIG. 3(d), a case where the blood flow velocity gradient distribution from the selected measurement point to the blood vessel wall surface is approximated by the primary straight line will be described.

FIG. 4 (a) illustrates a case where the measurement point is one point, and in a case where the blood flow velocity gradient distribution from the selected measurement point to the blood vessel wall surface is approximated by the primary straight line 41, although not illustrated in FIGS. 1 and 2, it is necessary to specify a position y_{w} of the blood vessel wall surface from the tomographic image formed by the tomographic image forming unit 151, such as a B mode image. At this time, as a specifying method of the position y_{w} of the blood vessel wall surface, computation may be automatically performed from the tomographic image, such as a B mode image formed by the tomographic image forming unit 151 and stored in the memory 16, or may be instructed by the examiner through the input unit 10. Since a definite integral of the blood flow velocity gradient is the blood flow velocity, the blood flow velocity at a measurement point y₀ is an area of a trapezoid ABCD, and the equation (3) is established.

$${u}_{0} = \frac{1}{2} ( {( \frac{du}{dy} )}_{y = {y}_{0}} + {( \frac{du}{dx} )}_{y = {y}_{w}} ) {y}_{0} \qquad ( 3 )$$

Here, μ₀ represents the blood flow velocity at the measurement point y₀ and du/dy represents the blood flow velocity gradient.

Therefore, the wall surface velocity gradient is calculated by the equation (4).

$${( \frac{du}{dy} )}_{y = {y}_{w}} = \frac{2 {u}_{0}}{{y}_{0}} - {( \frac{du}{dy} )}_{y = {y}_{0}} \qquad ( 4 )$$

In other words, the approximate model (primary straight line) is applied to the blood flow velocity gradient distribution in the vicinity of the blood vessel wall surface, and based on the relationship in which the definite integral of the blood flow velocity gradient distribution from the blood vessel wall surface to the selected measurement point becomes the same as the blood flow velocity at the selected measurement point, it is possible to calculate the estimated value of the wall surface velocity gradient from the values of the blood flow velocity and the blood flow gradient at the selected measurement point.

In addition, FIG. 4 (b) illustrates a case where the measurement points are two points, and the blood flow velocity gradient distribution from each of the measurement points to the blood vessel wall surface is approximated by the primary straight line 42. At this time, the position of the blood vessel wall surface is not necessarily specified. The relationship between the blood flow velocity and the blood flow velocity gradient at measurement points y₁ and y₂ is described by the equation (5) from the trapezoid ABCD and the trapezoid ABEF, with the coordinates y_{w} of the blood vessel wall surface being unknown.

$$\begin{matrix}{u}_{1} = \frac{1}{2} ( {( \frac{du}{dy} )}_{y = {y}_{1}} + {( \frac{du}{dy} )}_{y = {y}_{w}} ) ( {y}_{1} - {y}_{w} ) & ( 5 ) \\ {u}_{2} = \frac{1}{2} ( {( \frac{du}{dy} )}_{y = {y}_{2}} + {( \frac{du}{dy} )}_{y = {y}_{w}} ) ( {y}_{2} - {y}_{w} ) & \end{matrix}$$

When the equation (5) is solved as simultaneous equations, the wall surface velocity gradient (du/dy)_{y=yw} is calculated. For example, by substituting the measurement point y₁ with a y coordinate of the extreme value a and the measurement point y₂ with a y coordinate of the inflection point b into the equation (5), even when the position y_{w} of the blood vessel wall surface is unclear, the wall surface velocity gradient can be obtained.

<Step S8>

The wall shear stress operation unit 156 calculates the wall shear stress after receiving the value of the wall surface velocity gradient from the wall surface velocity gradient operation unit 155. The wall shear stress τ is given by equation (6).

$$τ = {μ ( \frac{du}{dy} )}_{y = {y}_{w}} \qquad ( 6 )$$

Here, μ is the viscosity coefficient of the blood.

<Step S9>

After receiving the tomographic image of the blood vessel 30 from the tomographic image forming unit, the Doppler velocity from the Doppler velocity extraction unit 152, the blood flow velocity distribution which is the distribution of the blood flow velocities in the direction parallel to the wall surface of the blood vessel 30 from the velocity distribution operation unit 158, the blood flow velocity gradient distribution from the velocity gradient distribution operation unit 153, the predetermined range from the range specifying operation unit 154, the value of the wall surface velocity gradient from the wall surface velocity gradient operation unit 155, and the information of the wall shear stress from the wall shear stress operation unit 156, the display image forming unit 157 forms the entirety or a part of the information as a display image in accordance with a predetermined format or an instruction input from the input unit 10.

In the apparatus configuration of the embodiment, the display unit 14 may display the blood flow velocity distribution calculated by the velocity distribution operation unit 158 and the blood flow velocity gradient distribution calculated by the velocity gradient distribution operation unit 153, and may display the predetermined range calculated by the range specifying operation unit 154 by superimposing the range on the blood flow velocity distribution or the blood flow velocity gradient distribution. For example, as illustrated in FIG. 5(a) or 5(b), with respect to the blood flow velocity distribution which is the distribution of the blood flow velocities in the direction parallel to the blood vessel wall surface or the blood flow velocity gradient distribution, the display image to be displayed by coloring the inside or the outside of the predetermined ranges 51 and 52 or by simply indicating the coordinates of the boundary of the predetermined ranges 51 and 52 as a numerical value is formed.

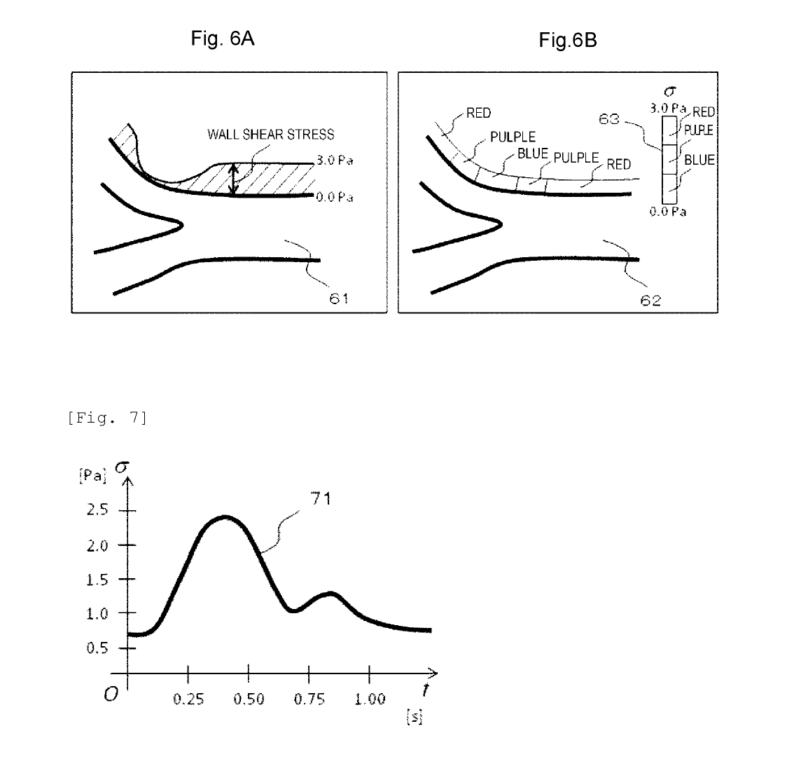

In addition, as illustrated in FIG. 6(a) or 6(b), the spatial distribution information of the wall surface velocity gradient or the wall shear stress calculated for the plurality of points is presented as perception information to the examiner together with the tomographic images 61 and 62 of the blood vessel. In other words, by superimposing the tomographic image of the blood vessel of the examination target formed by the tomographic image forming unit 151 on the information that becomes a diagnosis index of the examination target, such as the spatial distribution information of the wall surface velocity gradient or the wall shear stress, the superimposed image is displayed as a spatial distribution diagram.

At this time, as illustrated in FIG. 6(a), the magnitude of the wall shear stress may be indicated by the width as the perception information, or as illustrated in FIG. 6(b), the magnitude of the wall shear stress may be displayed being colored by using a color bar 63. In this manner, displaying by overlapping the tomographic image of the blood vessel, such as a B mode image, on at least one of the blood flow velocity distribution and the blood flow velocity gradient distribution, helps the examiner understand the spatial distribution of the information.

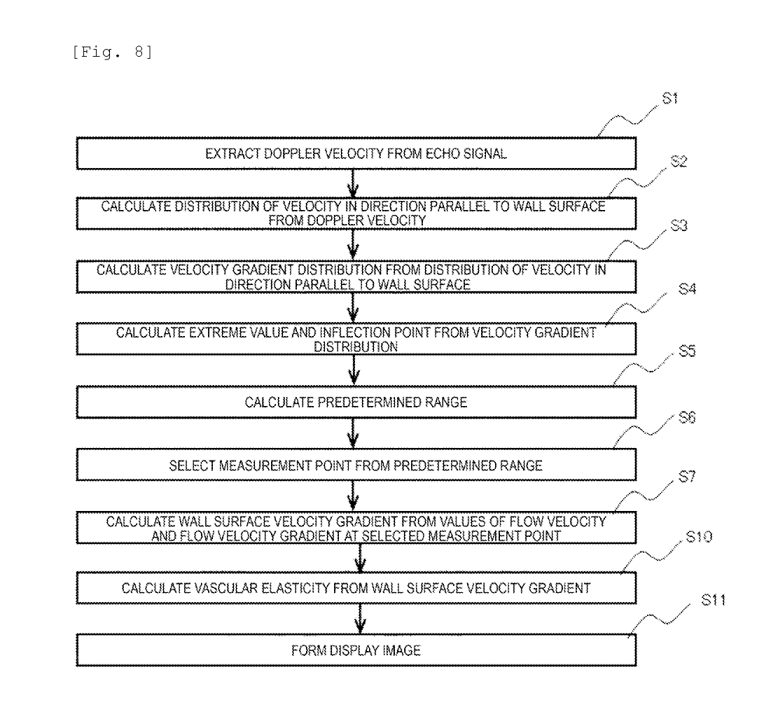

Further, as illustrated in FIG. 7, time-series change information 71 of the wall surface velocity gradient or the wall shear stress may be presented to the examiner. In the drawing, the horizontal axis represents time (s) and the vertical axis represents wall shear stress (Pa). In particular, displaying the information of the time-series change together with the heartbeat signal helps the examiner understand the correlation between the time-series change of the wall shear stress and the beat. In other words, the instruction is performed such that the heartbeat signal information of the examination target is input from the input unit 10, and the display unit 14 displays information, such as the wall shear stress that becomes the diagnosis index of the examination target together with the input heartbeat signal information as a time-series change. Furthermore, at least one of statistical values, such as maximum value, minimum value, average value, or medium value, of the spatial distribution information and the time-series change information of the wall shear stress may be presented to the examiner.

### Example 2

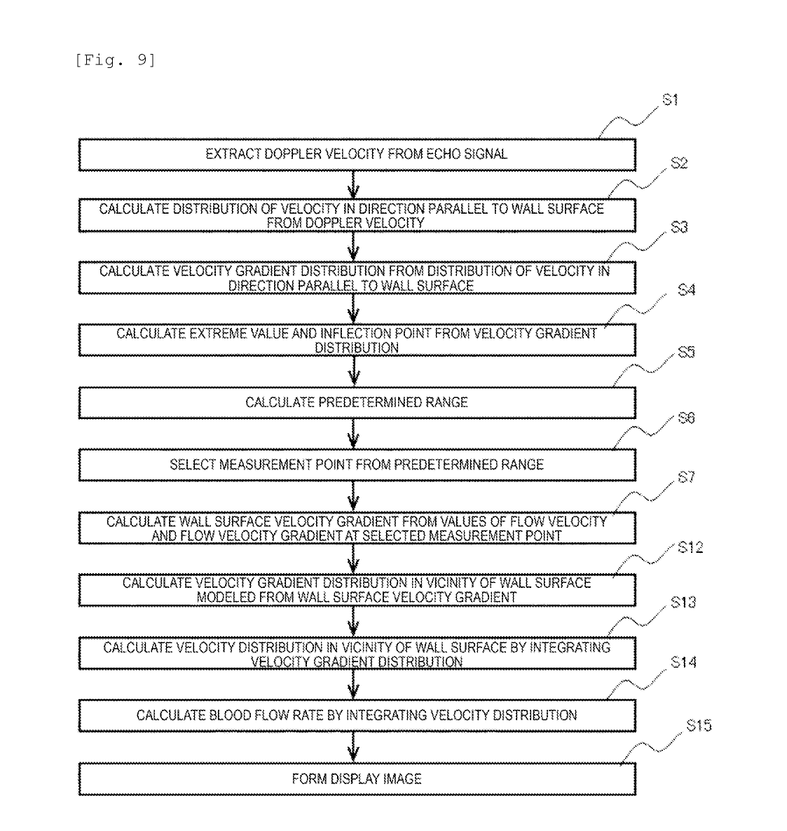

In Example 1, a configuration in which the wall shear stress operation unit that calculates the wall shear stress by using the estimated value of the wall surface velocity gradient is provided as a diagnosis index operation unit that calculates information which becomes a diagnosis index of the examination target is described, but the wall surface velocity gradient is also used for calculation of a vascular elasticity measurement method by the pressure gradient. As Example 2, an example of an ultrasonic imaging device including a vascular elasticity operation unit as the diagnosis index operation unit that calculates the information which becomes the diagnosis index of the examination target is described mainly focusing on points different from the ultrasonic imaging device of Example 1. Since a computation processing flow, becomes different after step S8, instead of steps S8 and S9 of FIG. 2, a flowchart in which steps S10 and S11 are inserted is illustrated in FIG. 8.

<Step S10>

In Example 1, the wall shear stress operation unit 156 receives the output of the wall surface velocity gradient operation unit 155, and the wall shear stress is calculated. In Example 2, although not illustrated in the drawing, the vascular elasticity operation unit is provided instead of the wall shear stress operation unit 156, and the vascular elasticity operation unit receives the output of the wall surface velocity gradient operation unit 155 of FIG. 1 and calculates the vascular elasticity. Further, the calculated vascular elasticity is stored in the memory 16. The vascular elasticity E is given by the equation (7).

$$E = \frac{2 μ LR}{h Δ d} {( \frac{du}{dy} )}_{y = {y}_{w}} \qquad ( 7 )$$

Here, R represents an inner diameter of the blood vessel, h represents the thickness of the blood vessel, L represents the distance between two points used for calculation of the pressure gradient, Δd represents the inner diameter difference between the two points, and μ represents the viscosity coefficient of the blood. As the parameters, for example, either a predetermined value, a value input from the input unit 10, or a value calculated from the echo signal may be adopted.

<Step S11>

In Example 2, after receiving the tomographic image of the blood vessel 30 from the tomographic image forming unit 151, the Doppler velocity from the Doppler velocity extraction unit 152, the blood flow velocity distribution which is the distribution of the blood flow velocities in the direction parallel to the wall surface of the blood vessel 30 from the velocity distribution operation unit 158, the blood flow velocity gradient distribution from the velocity gradient distribution operation unit 153, the predetermined range from the range specifying operation unit 154, the value of the wall surface velocity gradient from the wall surface velocity gradient operation unit 155, and the information of the vascular elasticity from the vascular elasticity operation unit, the display image forming unit 157 forms the entirety or a part of the information as a display image in accordance with a predetermined format or an instruction input from the input unit 10. A specific example of the display format conforms to step S9 in FIG. 2.

### Example 3

Example 3 is an example of an ultrasonic imaging device including a blood flow rate operation unit which serves as a diagnosis index operation unit that calculates information which becomes the diagnosis index of the examination target. The configuration of Example 3 will be described focusing on points different from the ultrasonic imaging device of Example 1. In the example, instead of the wall shear stress operation unit 156 of the apparatus configuration illustrated in FIG. 1, a flow rate operation unit is provided. The flow rate operation unit receives the output of the wall surface velocity gradient operation unit 155 and calculates the blood flow rate. Specifically, by using the value of the wall surface velocity gradient calculated by the wall surface velocity gradient operation unit 155, the flow rate operation unit substitutes the blood flow velocity distribution in the range from the blood vessel wall surface to the measurement point y1 by the approximate model (quadratic curve), and the blood flow rate is calculated by integrating the flow velocity distribution after the substitution.

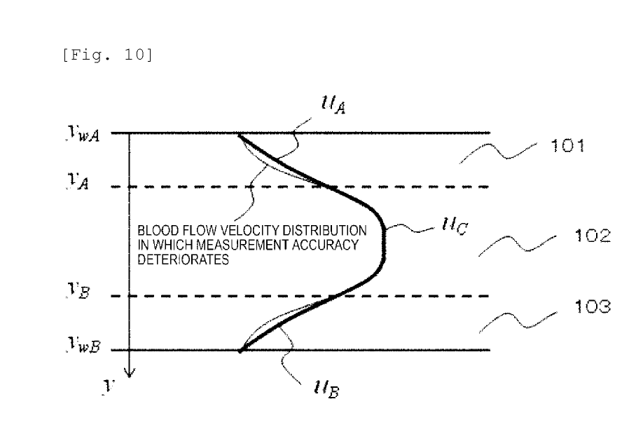

According to the configuration of the example, it is possible to obtain the blood flow rate with higher accuracy. FIG. 9 illustrates a flowchart in which steps S12, S13, S14, and S15 are inserted instead of steps S8 and S9 in FIG. 2 with respect to step S8 and the following steps thereof of which contents are different from those of Example 1 in the computation processing flow.

<Step S12>

The flow rate operation unit which is not illustrated in the drawing calculates the blood flow rate after receiving the numerical value of the wall surface velocity gradient. First, by using the value of the wall surface velocity gradient calculated by the wall surface velocity gradient operation unit 155, the approximate model of the blood flow velocity gradient distribution is given by the equation (8).

$$\frac{du}{dy} = {( \frac{du}{dy} )}_{y = {y}_{w}} + y \frac{{( \frac{du}{dy} )}_{y = {y}_{1}} - {( \frac{du}{dy} )}_{y = {y}_{w}}}{{y}_{1} - {y}_{w}} \qquad ( 8 )$$

<Step S13>

By integrating the equation (8), the approximate model of the blood flow velocity distribution is given by the equation (9).

$$u = {y ( \frac{du}{dy} )}_{y = {y}_{w}} + {y}^{2} \frac{{( \frac{du}{dy} )}_{y = {y}_{1}} - {( \frac{du}{dy} )}_{y = {y}_{w}}}{2 ( {y}_{1} - {y}_{w} )} \qquad ( 9 )$$

<Step S14>

By integrating the equation (9) over the entire blood vessel section, the blood flow rate can be obtained. FIG. 10 is a diagram for describing the integration range of the blood flow rate operation in the configuration of Example 3, 101 and 103 indicate a region integrating the modeled blood flow velocity distribution, and 102 indicates a region integrating the measured blood flow velocity distribution. As illustrated in FIG. 10, when u_{A} represents an approximate model of the blood flow velocity distribution in the vicinity of a blood vessel wall surface A in the region 101 integrating the modeled blood flow velocity distribution, u_{B} represents the approximate model of the blood flow velocity distribution in the vicinity of the blood vessel wall surface B in the region 103 integrating the modeled blood flow velocity distribution, and u_{C} represents the blood flow velocity distribution actually measured at the center of the blood vessel in the region 102 integrating the measured blood flow velocity, the blood flow rate is obtained by the following equation.

Q=∫_{y}_{_{wA}}^{y}^{_{A}}u_{A}dy+∫_{y}_{_{B}}^{y}^{_{wB}}u_{B}dy+∫_{y}_{_{A}}^{y}^{_{B}}u_{C}dy (10)

Here, y_{wA} represents coordinates of the blood vessel wall surface A, y_{wB} is coordinates of the blood vessel wall surface B, y_{A} is a measurement point in the vicinity of the blood vessel wall surface A, and y_{B} is a measurement point in the vicinity of the blood vessel wall surface B.

<Step S15>

In Example 3, after receiving the tomographic image of the blood vessel 30 from the tomographic image forming unit 151, the Doppler velocity from the Doppler velocity extraction unit 152, the blood flow velocity distribution which is the distribution of the blood flow velocities in the direction parallel to the wall surface of the blood vessel 30 from the velocity distribution operation unit 158, the blood flow velocity gradient distribution from the velocity gradient distribution operation unit 153, the predetermined range from the range specifying operation unit 154, the value of the wall surface velocity gradient from the wall surface velocity gradient operation unit 155, and the information of the blood flow rate from the blood flow rate operation unit, the display image forming unit 157 forms the entirety or a part of the information as a display image in accordance with a predetermined format or an instruction input from the input unit 10. A specific example of the display format conforms to step S9.

The ultrasonic imaging device of the present invention is not limited to the above-described embodiment, and addition and deletion of elements can be appropriately performed. For example, each example includes a wall shear stress operation unit, the vascular elasticity operation unit, or a blood flow rate operation unit, as a diagnosis index operation unit that calculates information which becomes the diagnosis index of the examination target, but it is also possible to have a configuration in which two or three of the units are combined with each other. In addition, although Example 1 has been described with the early diagnosis of arteriosclerosis as an example, this is not limited to the artery to which the present invention is applied, and it is also possible to apply the present invention to the blood flow velocity gradient measurement in a vein, such as a lower extremity vein. The lower extremity vein is a site at which thrombus and varicose veins are likely to occur, and the present invention may also be applied to these diagnoses.

### REFERENCE SIGNS LIST

1 apparatus main body
2 ultrasonic probe
3 living body
10 input unit
11 control unit
12 transmission unit
13 reception unit
14 display unit
15 signal processing unit
16 memory
30 blood vessel
41, 42 primary straight line
51, 52 predetermined range
61, 62 tomographic image of blood vessel
63 color bar
71 time-series change information
101, 103 region integrating modeled blood flow velocity distribution
102 region integrating measured blood flow velocity distribution
151 tomographic image forming unit
152 Doppler velocity extraction unit
153 velocity gradient distribution operation unit
154 range specifying operation unit
155 wall surface velocity gradient operation unit
156 wall shear stress operation unit
157 display image forming unit
158 velocity distribution operation unit

1. An ultrasonic imaging device comprising:
a receiving unit that receives an echo signal reflected by an examination target; and
a signal processing unit that processes the echo signal received by the receiving unit,
wherein the signal processing unit includes
a velocity gradient distribution operation unit that calculates a blood flow velocity gradient distribution from a value of a blood flow velocity, in a direction parallel to a blood vessel wall surface of the examination target, calculated from the echo signal at a plurality of measurement points arranged in a radial direction from the blood vessel wall surface toward the center of a blood vessel,
a range specifying operation unit that calculates a predetermined range in the blood flow velocity gradient distribution, and
a wall surface velocity gradient operation unit that calculates an estimated value of a blood flow velocity gradient on the blood vessel wall surface from values of the blood flow velocity and the blood flow velocity gradient at the measurement points within the predetermined range.

2. The ultrasonic imaging device according to claim 1,
wherein the range specifying operation unit determines a region to be rejected based on a shape of the blood flow velocity gradient distribution, and calculates the predetermined range as a range excluding the region to be rejected with respect to the blood flow velocity gradient distribution.

3. The ultrasonic imaging device according to claim 2,
wherein the range specifying operation unit calculates the predetermined range with reference to at least any of a point at which a monotonous increase turns into a monotonous decrease from the blood vessel wall surface toward the center of the blood vessel and an inflection point at which an upward convex turns into a downward convex, in the blood flow velocity gradient distribution.

4. The ultrasonic imaging device according to claim 3,
wherein the range specifying operation unit sets a boundary of a predetermined range of the measurement points used for estimation of the blood flow velocity gradient on the blood vessel wall surface as the point at which the monotonous increase turns into the monotonous decrease from the blood vessel wall surface toward the center of the blood vessel, and
wherein the wall surface velocity gradient operation unit calculates the estimated value of the blood flow velocity gradient on the blood vessel wall surface from the values of the blood flow velocity and the blood flow velocity gradient at at least one or more the measurement points within the predetermined range including the boundary.

5. The ultrasonic imaging device according to claim 4,
wherein the wall surface velocity gradient operation unit calculates the estimated value of the blood flow velocity gradient on the blood vessel wall surface from the values of the blood flow velocity and the blood flow velocity gradient at at least any of the point at which the monotonous increase turns into the monotonous decrease from the blood vessel wall surface toward the center of the blood vessel and the inflection point at which the upward convex turns into the downward convex.

6. The ultrasonic imaging device according to claim 1,
wherein the signal processing unit includes a diagnosis index operation unit that calculates information which becomes a diagnosis index of the examination target by using the estimated value of the blood flow velocity gradient, on the blood vessel wall surface, calculated by the wall surface velocity gradient operation unit.

7. The ultrasonic imaging device according to claim 6,
wherein the diagnosis index operation unit is a wall shear stress operation unit that calculates a wall shear stress from the estimated value of the blood flow velocity gradient on the blood vessel wall surface.

8. The ultrasonic imaging device according to claim 6,
wherein the diagnosis index operation unit is a vascular elasticity operation unit that calculates a vascular elasticity from the estimated value of the blood flow velocity gradient on the blood vessel wall surface.

9. The ultrasonic imaging device according to claim 6,
wherein the diagnosis index operation unit is a blood flow rate operation unit that calculates a blood flow rate from the estimated value of the blood flow velocity gradient on the blood vessel wall surface.

10. The ultrasonic imaging device according to claim 1, further comprising:
a display unit that displays information obtained by the signal processing unit,
wherein the display unit displays the blood flow velocity gradient distribution calculated by the velocity gradient distribution operation unit, and
wherein the display unit displays the predetermined range calculated by the range specifying operation unit with being superimposed on the blood flow velocity gradient distribution.

11. The ultrasonic imaging device according to claim 1,
wherein the signal processing unit includes a tomographic image forming unit that forms a tomographic image of the examination target from the echo signal, and
wherein the wall surface velocity gradient operation unit calculates the estimated value of the blood flow velocity gradient on the blood vessel wall surface from positional information of the blood vessel wall surface specified from the tomographic image and the values of the blood flow velocity and the blood flow velocity gradient at the measurement points within the predetermined range calculated by the range specifying operation unit.

12. The ultrasonic imaging device according to claim 6, further comprising:
a display unit that displays the information obtained by the signal processing unit,
wherein the signal processing unit includes a tomographic image forming unit that forms the tomographic image of the examination target from the echo signal, and
wherein the display unit displays information that becomes a diagnosis index of the examination target with being superimposed on the tomographic image, as a spatial distribution diagram.

13. The ultrasonic imaging device according to claim 6, further comprising:
an input unit that inputs heartbeat signal information of the examination target; and
a display unit that displays the information obtained by the input unit and the signal processing unit,
wherein the display unit displays information that becomes a diagnosis index of the examination target together with the heartbeat signal information of a time-series change.

14. An operation method in an ultrasonic imaging device, the method comprising:
a step of extracting a blood flow velocity component in an ultrasonic irradiation direction in a blood vessel of an examination target from an echo signal reflected by the examination target;
a step of calculating a blood flow velocity distribution which is a distribution of blood flow velocities in a direction parallel a blood vessel wall surface at a plurality of measurement points arranged in a radial direction from the blood vessel wall surface toward the center of a blood vessel, based on the blood flow velocity component;
a step of calculating a blood flow velocity gradient distribution from the blood flow velocity distribution by a differential operation;
a step of calculating a predetermined range in the blood flow velocity gradient distribution;
a step of selecting one or more measurement points among the measurement points within the predetermined range; and
a step of calculating an estimated value of a blood flow velocity gradient on the blood vessel wall surface from values of a blood flow velocity and a blood flow velocity gradient at the selected measurement points.

15. The operation method according to claim 14,
wherein, by using the value of the blood flow velocity gradient at the selected measurement points, an approximate model is employed to the blood flow velocity gradient distribution in the vicinity of the blood vessel wall surface, and the estimated value of the blood flow velocity gradient on the blood vessel wall surface is calculated from the values of the blood flow velocity and the blood flow velocity gradient at the selected measurement points based on a relationship in which a definite integral of the blood flow velocity gradient distribution is equal to the value of the blood flow velocity at the selected measurement points in the approximate model.

16. The operation method according to claim 15,
wherein the approximate model of the blood flow velocity gradient distribution in the vicinity of the blood vessel wall surface is set to be a primary straight line.
